(19) United States
(12) Patent Application Publication
(10) Pub. No.: US 2009/0007904 A1
(43) Pub. Date: Jan. 8, 2009

(54) Device and Method for Generating an Aerosol From a Liquid Formulation and Ensuring Its Sterility

(75) Inventors: Jeffrey A. Schuster, Oakland, CA (US); Peter Holst, Hayward, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: ARADIGM CORPORATION, Hayward, CA (US)

(21) Appl. No.: 11/576,441

(22) PCT Filed: Oct. 12, 2005
(86) PCT No.: PCT/US2005/036755

§ 371 Date: Jul. 21, 2008

Related U.S. Application Data
(60) Provisional application No. 60/618,344, filed on Oct. 12, 2004.

(51) Int. Cl. A61M 11/00

(52) U.S. Cl. 128/200.23

(57) ABSTRACT

A drug delivery device containing a sterile multi dose reservoir. Said sterile reservoir can be used with many types of delivery including injectors or aerosol drug delivery systems. Elevated pressure surrounding the reservoir is used during storage to ensure sterility is maintained. Mechanisms to prevent delivery in the case of potential compromise of sterility are disclosed. A device using the pressure to meter formulation from the reservoir is disclosed.

### CROSS-REFERENCE

This application is a 371 National Phase of International Application Serial No. PCT/US2005/036755, filed Oct. 12, 2005 which claims priority to U.S. Provisional Patent Application Serial No. 60/618,344 filed Oct. 12, 2004, which are incorporated herein by reference in their entirety noting that the current application controls to the extent there is any contradiction with any earlier applications and to which applications we claim priority under 35 USC § 120.

### FIELD OF THE INVENTION

The present invention relates to methods of storing liquid drug formulations, and presenting them for delivery to a human or animal, preferably by aerosol delivery. Methods are described for maintaining the formulations in a sterile state, and for notifying the user or locking out the delivery to the user if the sterility is compromised.

### BACKGROUND OF THE INVENTION

The production of finely dispersed aerosols is important for aerosolized delivery of drugs to obtain of the aerosolized particles to the respiratory tract of humans or animals. Many aerosol drug delivery systems generate aerosol particles at the time of use from a reservoir containing multiple doses of liquid formulation. One example of such a device is described in U.S. Pat. No. 5,497,944. Other technologies that can be adapted to this type of delivery are described in U.S. Pat. Nos. 6,119,953 and 6,174,469, and U.S. patent application Ser. Nos. 09/591,365 and 10/649,376, incorporated here in their entirety by reference. Because the aerosolization technology used in these and similar inventions is somewhat costly, it is preferable to use them for the delivery of multiple, rather than single, doses. Similarly, reduced cost can be achieved by using a multidose reservoir. Simplicity in the mechanism that meters the dose from this reservoir is preferred.

Because these technologies are optimized for efficient delivery of the formulation to the lung, it is a problem that any infective agent such as bacteria or viruses that are contained in the formulation prior to aerosolization and delivery will also be delivered to the lung, leading to the possibility of lung or systemic infection. Lung infections can be caused by, for example, Pseudomonas aeruginosa, Mycobacterium tuberculosis, Pneumocystis, and Legionella.

The US Department of Health and Human Services Food and Drug Administration Center for Drug Evaluation and Research (CDER) released a guidance for industry in July 2002 entitled “Nasal Spray and Inhalation Solution, Suspension, and Spray Drug Products—Chemistry, Manufacturing, and Controls Documentation”. This guidance states that “For device-metered, aqueous-based inhalation spray drug products . . . studies should be performed to demonstrate the appropriate microbiological quality through the life of the reservoir and during the period of reservoir use. Such testing could assess the ability of the container closure system to prevent microbial ingress into the formulation and/or the growth inhibiting properties of the formulation.” It is thus now a regulatory requirement in the United States that aqueous based inhalers be sterile or bacteriostatic through life.

One solution is to include preservatives, such as benzylkonium chloride, in the formulation. However preservatives can lead to lung irritation, and may not be effective against all microorganisms.

A preferable solution is to maintain the sterility of the drug reservoir through mechanical means, and to deliver a preservative free formulation. One way of ensuring sterility is in the use of pressure gradients. For example, pharmaceutical products are usually manufactured in a sterile area. In addition to air filtration and gowning procedures, sterility is maintained in these areas by maintaining them at a higher air pressure than surrounding areas. This ensures that any leak has flow out from the sterile area, eliminating the possibility of ingress of pathogens.

### SUMMARY OF THE INVENTION

A drug delivery device comprising a sterile multi-dose reservoir wherein the sterile reservoir can be used in combination with a range of delivery devices including injectors and aerosol drug delivery devices. The device utilizes a chamber or plenum which is maintained in an elevated pressure and surrounds the reservoir. The device includes components which prevent delivery of the drug and/or provides a warning when sterility is compromised. Valves may be used to meter formulation from the reservoir and thereby create a sterile stream of formulation from the reservoir which can be used to create an aerosol or for injection.

Drug delivery devices disclosed comprised of a container of pressurized gas. The container is removably, or preferably permanently, placed within the device. The container or the device has a metering valve which releases a metered amount of gas from the container upon actuation. The device also include a reservoir which is loaded with a formulation such as a liquid solution or suspension comprising of a pharmaceutically acceptable carrier and a pharmaceutically active drug. A channel such as a capillary tube leads from the reservoir and a one-way valve may be in the channel and may include an aerosolization nozzle at the end of the channel. A chamber is in physical contact with the reservoir and in gas flow connection with the container of pressurized gas. When the pressurized gas is released from the metering valve the chamber is pressurized and compresses flexible walls of the reservoir thereby expelling formulation from the reservoir at a predetermined rate of delivery and provide a predetermined dose amount which may be in an aerosol.

According to a first aspect of the invention, there is provided a device for delivering a metered quantity of a drug product from a reservoir to an aerosolization means. This device comprises:
(a) a pressurized gas source;
(b) a valve which meters out a predetermined amount of gas from the gas source;
(c) A reservoir which can be loaded with a formulation comprising a pharmaceutically active drug
(d) a plenum around the reservoir;
(e) a first fluid channel for delivering a portion or all of the metered gas to the plenum; and
(f) a second fluid channel for delivering, under the exertion of the gas pressure in the plenum, a predetermined amount of the formulation contained in the reservoir to a component such as a nozzle which aerosolizes the formulation.

In a preferred embodiment, the pressurized gas is additionally used as the power source for creating an aerosol out of the formulation.

The device may incorporate a means (such a docking unit) for the removal and replacement of the pressurizing gas source and/or the drug reservoir. In a preferred embodiment, the amount of drug product in the reservoir and the amount of gas that can be delivered from the gas source are chosen such that they both last for essentially the same number of doses, and after the doses are expended, the entire system is disposed of.

It is a second aspect of the invention, after the predetermined amount of drug formulation is expelled at a first pressure, the pressure in the plenum falls to a second pressure greater than the surrounding ambient pressure due to flow of gas through a venting means. At said second pressure the means for venting the gas and reducing the pressure is closed by a vent closing means, and the second pressure is essentially maintained in the plenum. This has the effect of:
(a) ensuring that the venting means is open only when the pressure is above the second pressure, preventing any ingress of pathogens into the drug reservoir during a dosing event, and
(b) ensuring that the plenum surrounding the drug reservoir is pressurized at a pressure above the ambient pressure during storage between doses, so that any leaks in the plenum or the seal of the vent closing means will flow outward from the plenum and drug reservoir, preventing any ingress of pathogens during storage.

The venting means could be any type of valve or an orifice of any shape or aspect. In a preferred embodiment, the venting means is an integral part of the atomizer, and the process of venting is an integral part of the atomization process. The vent closing means can be any manner of seal, cover, cap, or the like. It can be actuated independently of the described invention, for example by a timer and actuating means such as a motor, spring, or the like. Preferably, the valve or vent closing means is opened by gas pressure in the plenum, opening at some pressure between the first pressure and second pressure, and closing again at the second pressure.

It is a third aspect of the invention to provide a means for preventing the delivery of the medication if the sterility of the formulation has potentially been compromised. This means would be activated if the pressure fell below a third pressure, said third pressure being less than the second pressure, and higher than the surrounding ambient pressure. This could be accomplished with an electronic component, utilizing a pressure transducer and electronics. Preferably, it is accomplished with a mechanical component that is responsive to the pressure in the plenum. This mechanical component could be a stand alone sub-system, but is preferably incorporated into the vent closing component. The mechanism for preventing delivery could be realized in many ways, including but not limited to notifying the user of the potential for lack of sterility, or by locking out the use of the device.

These and other objects, advantages, and features of the invention will become apparent to those persons skilled in the art upon reading the details of the devices and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic overview of one embodiment of the invention, incorporated into a drug delivery system.

FIG. 2 is a schematic of one embodiment of the invention for delivering a predetermined amount of formulation from the reservoir.

FIG. 3 is a schematic of one embodiment of the system for ensuring sterility of the reservoir, shown in the stored, sterile state.

FIG. 4 is a schematic of one embodiment of the system for ensuring sterility of the reservoir, shown in the pressurized, delivery state.

FIG. 5 is a schematic of one embodiment of the system for preventing the delivery of the formulation in the event that the sterility has potentially been compromised, shown in the sterility compromised state.

FIG. 6 is a schematic of one embodiment of the system for notifying the user in the event that the sterility has potentially been compromised, shown in the sterility compromised state.

FIG. 7 is a schematic of a system that was implemented to use a pneumatic timer to control the amount of aerosol.

FIG. 8 is a graph of gas and liquid pressure, and liquid flow rate and duration achieved with the system of FIG. 7.

FIG. 9 is an alternate embodiment wherein sterility is maintained through the use of a one way valve in the capillary.

### DETAILED DESCRIPTION OF THE INVENTION

Before the present devices, formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Ambient pressure is defined as the absolute pressure of the air surrounding the device and the user at the time the invention is used or stored. More specifically, the ambient pressure will be understood to mean the maximum ambient pressure that might be expected to be encountered during the lifetime of the device population. For example, the elevation of the Dead Sea is 1286 feet below sea level. The highest pressure ever observed in this area 1.0818 bar.

Atomization, atomization component, atomizer, and the like, are used interchangeably and shall be interpreted to mean any of the numerous methods that are presently available, or may be invented in the future to generate an aerosol. Examples include, but are not limited to, vibrating meshes, jet nebulizers, extrusion through a nozzle, delivery of multiple fluids through a nozzle as disclosed in U.S. patent application Ser. No. 10/649,376, spinning tops, ultrasonic nebulizers, dry powder dispersers, condensation aerosol generators, electro-hydrodynamic aerosol generators, and extrusion through a nozzle in the form of a porous membrane as taught in U.S. Pat. No. 6,123,068 and other devices disclosed in patents and publications cited there all of which are incorporated here by reference, and the like.

Formulation shall mean any liquid, solid, powder, gel or other state of matter that can be atomized. Preferred formulations are liquid formulations which may be solutions and/or suspensions. Formulations include but are not limited to those comprising excipients that are suitable for pulmonary administration or injection, and comprise one or more active pharmaceutical ingredients.

Pneumatic timer shall mean a mechanism for timing an event wherein the source of energy is gas pressure.

Metering valve shall mean a mechanism for delivering a fixed, known amount of gas by measuring it out of a known volume. The volume can contain the gas, but preferably contains a liquid, which when released from the metering valve turns into a gas. An example is a metered dose inhaler, wherein the dose of a drug and a liquid propellant are controlled by a metering valve.

Capillary shall mean a channel for transport of a substance. The channel may be a tube with any diameter and cross section, although it is preferably a circular cross section. It can also be of varying or of constant cross sectional area, including a tapered cross section. The substance can be any substance capable of transport down the tube, but preferably contains at least one pharmaceutically active substance in liquid form in a tube of 1 mm in diameter or less e.g. 0.01 to 0.05 mm in diameter. It can be a gas or dry powder, but is preferably a liquid, wherein the at least one pharmaceutically active substance is in solution or suspension.

### EMBODIMENTS OF THE FIGURES

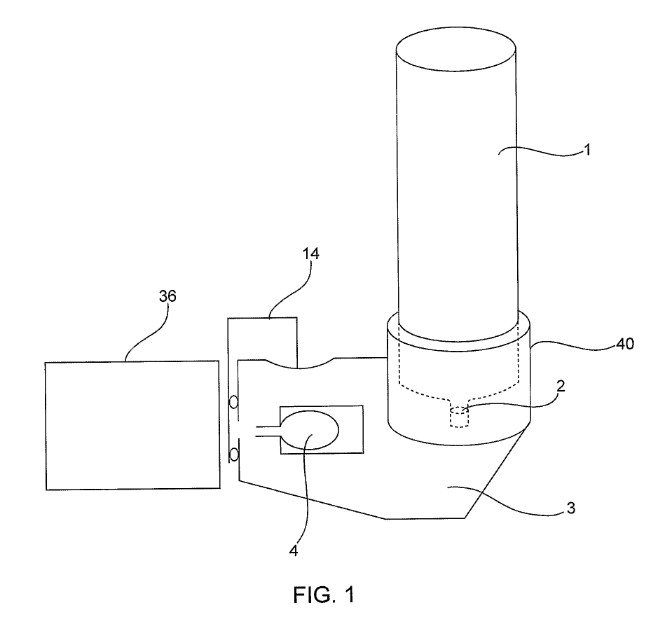

FIG. 1 shows an embodiment of an aerosol drug delivery system utilizing an embodiment of the invention. An air tight compressed gas source 1 contains the liquid, gas, or solid used to generate a gas which provides energy to the device, e.g. forces liquid from a reservoir 4 and interacts with the liquid as it passes through orifice (note: orifice needs to be labeled in FIG. 1) to create an aerosol. Many different methods could be used to generate the gas, including physical force (e.g. from a piston or cam) and chemical reactions. However, it is preferred to use a pressurized gas, or more preferably a high vapor pressure liquid, e.g. a low boiling point propellant which is liquid in the canister becomes gaseous on release to the chamber or plenum 3.

The gas from the source or canister 1 in this embodiment is inhaled by the user and thus needs to be a non-toxic, dust free, sterile, medical grade gas. Preferred pressurized gasses include air, argon, helium, or more preferably nitrogen. High vapor pressure liquids are preferred, because they maintain constant pressure as the contents of the gas source are depleted. Because higher pressures in this embodiment achieve smaller particles and larger delivered doses, relatively high vapor pressure liquids, including but not limited to liquid forms of CO₂ or NO₂, which are readily available as medical grade products in metal cylinders are more preferred. For lower dose or larger particle size products, other lower vapor pressure liquids, including but not limited to hydro-fluoro-alkanes (HFAs) or Chloro-Fluoro-Carbons (CFCs) could be used. Both are used extensively for inhalation products, although HFAs are preferred due to their lower potential for ozone depletion. Differing amounts of liquid, gas, or solid could be contained in the gas source, depending on the dose to be delivered, number of doses, and particle size desired. However, it is preferred that the gas source contain 2-50 gms of material, more preferable 5 to 25 grams, most preferably 8-16 gms of liquid, e.g. liquid CO₂ which vaporizes on release from the metering valve 2 of the canister 1.

In fluid contact with the gas source or canister 1 is a metering valve 2. This valve 2 is similar to metering valves currently in use for pressurized metered dose inhalers (pMDIs). There are numerous ways to actuate the metering valve 2, including pressing down on gas source 1 so that an end portion of the source 1 is moved toward and mechanically displaces and opens the metering valve 2. Other methods include, but are not limited to, mechanical and electronic breath actuation.

Because dosing reproducibility is important, the reproducibility of metering valve 2 must be such that 90% of actuations meter out an amount within ±25% of the target amount, preferably within ±15% of the target amount, still more preferably within ±5% of the target amount when the valve is repeatably actuated. Alternatively, metering valve 2 may be replaced by a mechanism for controlling a chemical reaction to generate a predetermined amount of gas. Alternatively, the amount of gas can be metered by a timing means that controls the amount of time that the pressurized gas is delivered to the system. The timing component could be but is not limited to a mechanical timer, or an electronic timer. Preferably, the timing means is a pneumatic timer.

The canister 1 may be a permanent part of the device. However, the canister 1 is possibly a disposable unit inserted into the docking unit 40 and placed in a position such that it has a gas tight connection with the chamber 3. The device can be sold without a canister in place and canisters can be sold separately. The canister may be designed to have only enough gas to expel all of the formulation from the reservoir 4. Alternatively, the canister may have sufficient gas to expel all of the formulation from several reservoirs so that the canister can be removed from the docking chamber 40 and placed within a device with a fully charged reservoir 4.

Upon metering of the gas source, the metering valve releases gas into plenum 3, causing the internal volume of the plenum or chamber 3 to increase in pressure. By controlling the volume of the plenum 3 and the amount of gas metered, any pressure up to the pressure equal to that within gas source 1 can be achieved. Fully contained within plenum 3 and surrounded by gas is a flexible reservoir 4. Mechanism 5 is used to seal off plenum 3 following a delivery event. The aerosol is generated into and delivered to the patient through mouth piece 36.

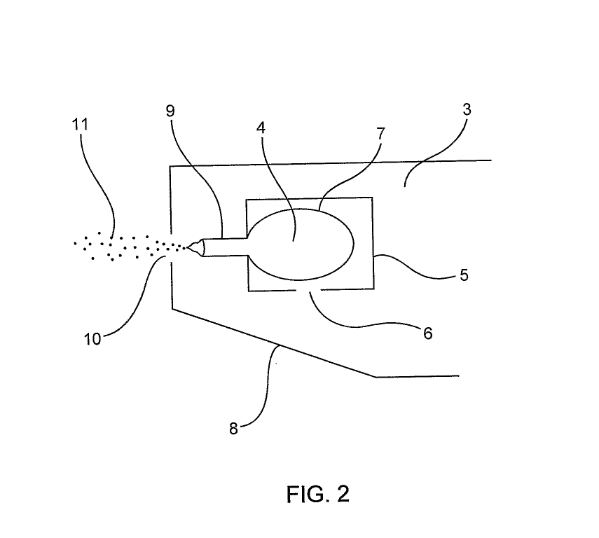

FIG. 2 shows a schematic of one embodiment of the method of using the gas pressure to meter a pre-determined amount of formulation from reservoir 4 to create aerosolized particles 11. In reservoir 4, the liquid formulation is contained within a flexible container 7, which is itself contained within a housing 5. Housing 5 is in fluid communication with the pressurized gas contained in plenum 3 via opening 6. Flexible container 7 can be implemented in many ways, including but not limited to a balloon bladder bellows, diaphragm, piston/cylinder, or the like. Preferably it is a polymer, foil or a laminate thereof with a degree of flexibility. Many different materials could be used for flexible container 7, so long as they have acceptable properties that do not impact the formulation adversely, including low extractables. Preferred materials include polyethelene, Cyclo Olefin Copolymers (COCs) and the like for drug contact, Polychlorotrifluoroethylene Chlorotrifluoroethene (PCTFE) or a foil such as aluminum for vapor barrier properties, and polymers such as nylon or polyester for mechanical strength.

When plenum 3 is pressurized, housing 5 will also be pressurized via opening 6. This pressure will compress flexible container 7 and drive the liquid formulation though capillary 9. The liquid formulation is then focused toward orifice 10, and the process of gas and liquid flow toward and through orifice 10 forms an aerosol 11.

One side of plenum 3, side 8, can be inwardly profiled or otherwise shaped such that the gas velocity v outside of opening 6 is reduced from the pressure the gas would have in plenum 3 in the absence of flow by the amount ½ ρv2, but greater than the surrounding ambient pressure and greater than the pressure at the exit of capillary 9. Alternative ways of achieving the desired pressure include the use of a venturi, or a pressure regulator.

By the proper choice of the position and area of opening 6, gas velocity outside of opening 6, stiffness of flexible container 7, viscosity of the formulation, and length and interior cross-section of capillary 9, the amount and rate of delivery of the formulation can be controlled. It is preferred not to include additives in the formulation to alter the viscosity. Preferably the container 7 is flexible enough, and the opening 6 is large enough, that the rate and amount of formulation delivered is largely set by the position of opening 6, the gas velocity outside of opening 6, and the dimensions of capillary 9.

Capillary 9 can have any shape, but is preferably of constant cross section (a cylinder) and more preferably is a right circular cylinder. At the exit of capillary 9, the cross sectional area is preferably 0.001 to 1 mm², more preferably 0.01 to 0.1 mm², most preferably 0.01 to 0.05 mm². The length of capillary 9 is preferably less than 25 mm, more preferably less than 12 mm, most preferably less than 6 mm.

The viscosity of the formulation is preferably 1 to 50 centipoise, more preferably 1 to 10 centipoise, most preferably 1 to 5 centipoise. The distance from the opening 6 to the orifice 10 is preferably 1 to 50 mm, more preferably 5 to 25 mm, most preferably 10 to 20 mm. The rate of delivery is preferably 0.1 to 500 μL/s, more preferably 1 to 250 μL/s, most preferably 3 to 100 μL/s.

Any number of orifice/capillary pairs can be used simultaneously, each of which having the above properties. Any pharmaceutically acceptable carrier can be used in the formulation, although it preferably comprises ethanol or ethanol/water mixtures, and more preferably comprises water. Preferably the drug is in solution, although it can also be in suspension. Poorly soluble compounds can be placed in solution using various additives, including but not limited to cyclodextrins. The amount of drug in the carrier is preferably in the range of 0.1 to 500 mg/mL, more preferably in the range of 1 to 100 mg/mL, Most preferably in the range of 10 to 75 mg/mL.

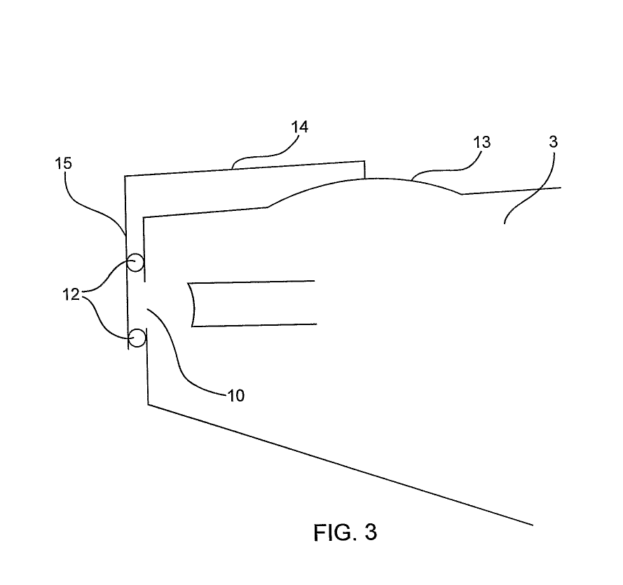

FIG. 3 shows an embodiment of the mechanism to ensure the sterility of the formulation on storage between doses, here shown in the closed, stored state. Diaphragm 13 or other component movable in response to a pressure change is in contact and responsive to the pressure in plenum 3. When the pressure in plenum 3 drops from the first pressure during delivery to the second pressure, diaphragm 13 pulls cover 15 over orifice 10 through linkage 14.

Seal 12 ensures a pressure tight fit for a sufficiently long time that the pressure is maintained between doses. The seal 12 may be comprised of a flexible ring of polymeric material shown in cross-section in FIGS. 1 and 3. It is preferable that the second pressure is relatively different (e.g. 2, 3 or 4 or more times greater) from the first pressure in order that the displacement of diaphragm 13 is maximized. It is preferable that the second pressure is minimized such that the amount of leakage and the requirements for seal 12 is minimized. The second pressure is preferably less than 50 bar, more preferably less than 10 bar, most preferably less than 5 bar. The pressure is preferably maintained at an acceptable level for at least one day, more preferably for at least one week, most preferably for at least one month. The device could be shipped and stored prior to use in this pressurized condition to ensure stability, but it is preferable to store and ship it in a sterile over-wrap prior to use, in an un-pressurized state.

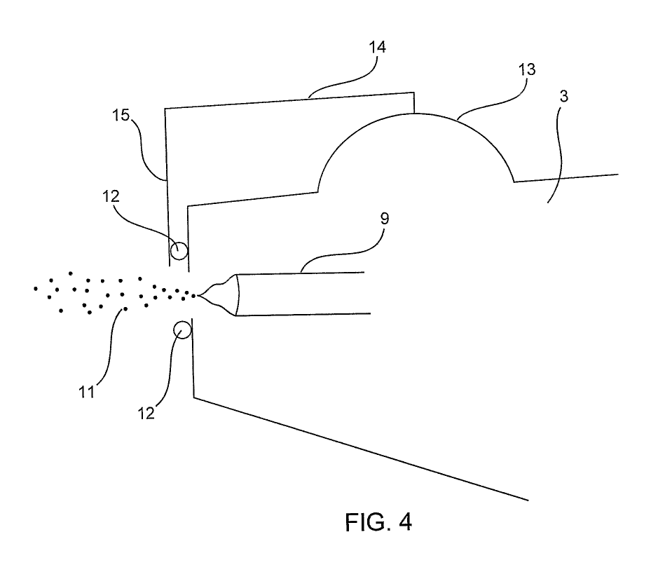

FIG. 4 shows the invention while the aerosol 11 is being generated. Because of the higher first pressure in plenum 3, diaphragm 13 is distended such that cover 15 is moved outward so as to uncover orifice 10, allowing the flow of gas and liquid, and the outward flow of the aerosol 11. The first pressure is preferably more than 2 bar, more preferably more than 10 bar, and most preferably more than 25 bar. In one preferred embodiment, the gas is CO₂ and the pressure is 25-70 bar.

Although the actuating means is shown here schematically as a diaphragm 13, other actuators responsive to the pressure in plenum 3, including a bellows, a piston with a return spring (mechanical or gas), a pressure transducer and electromechanical means, and the like, could be used.

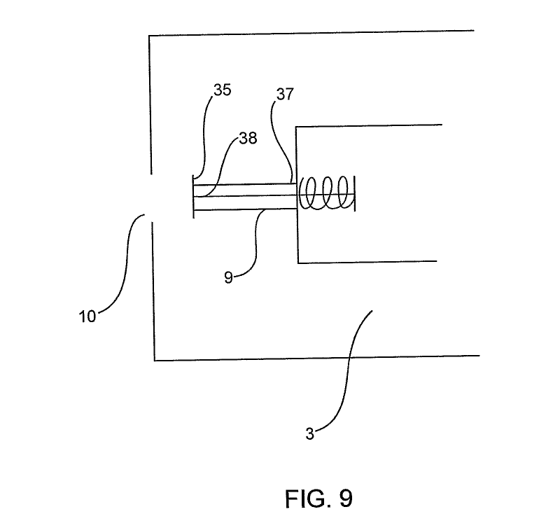

FIG. 9 shows a simpler embodiment of the invention wherein the diaphragm 13, linkage 14, cover 15, and seals 12 of the embodiment of FIG. 4 are replaced with a mechanical one way valve 35 in the capillary 9. The one way valve 35 could be placed anywhere along capillary 9, including the entrance 37 to capillary 9, but is preferably placed at the exit 38 of capillary 9 to ensure sterility along the entire length of capillary 9. The one way valve 35 allows the flow of formulation when the formulation is at a first pressure, and closes and prevents the ingress of contaminant when the formulation pressure is dropped to a second pressure which is less than the first pressure. With this one way valve 35, the liquid in the reservoir is maintained in a sterile state in much the same way as described above, as the one way valve 35 only opens when the formulation is pressurized, preventing inflow. However, it has the disadvantage that the interior of plenum (3) is not maintained in a sterile state.

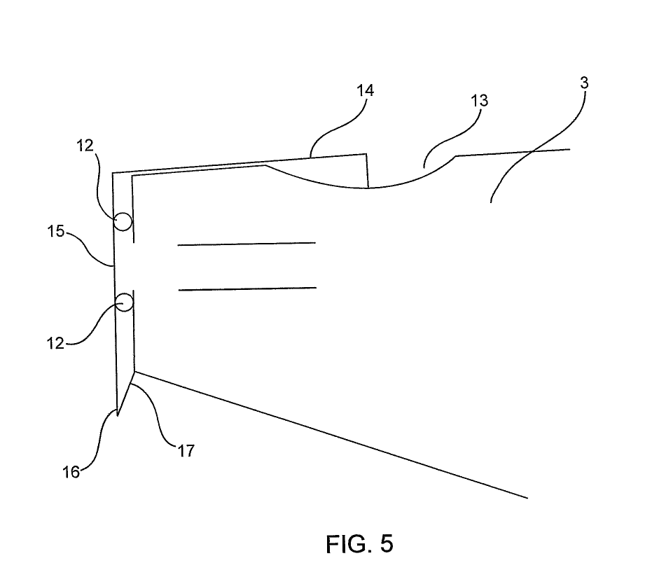

FIG. 5 shows schematically one embodiment of the mechanism to lock out use of the device in the event that the sterility may have been compromised, as could occur if there is a large leak, if seal 12 fails, or if the device is left for an unexpectedly long time without being used. When the pressure drops below a pre-determined third pressure which is less than the second pressure, diaphragm 13 moves cover 15 to a location such that locking elements 16 and 17 engage, locking out further actuation of the device. Diaphragm 13 could be a bi-stable device, wherein it transitions from a concave to a convex configuration at the third pressure, increasing the amount of movement available for cover 15.

In another embodiment, when the pressure drops to the third pressure, the metering valve (2 as shown in FIG. 1) is locked out such that the canister (1 of FIG. 1) cannot be depressed. Numerous other embodiments could be used, including a pressure transducer and electromechanical lock out means. This invention has the additional benefit that if the device passes its expiry date significantly due to lack of use, the device will be no longer usable.

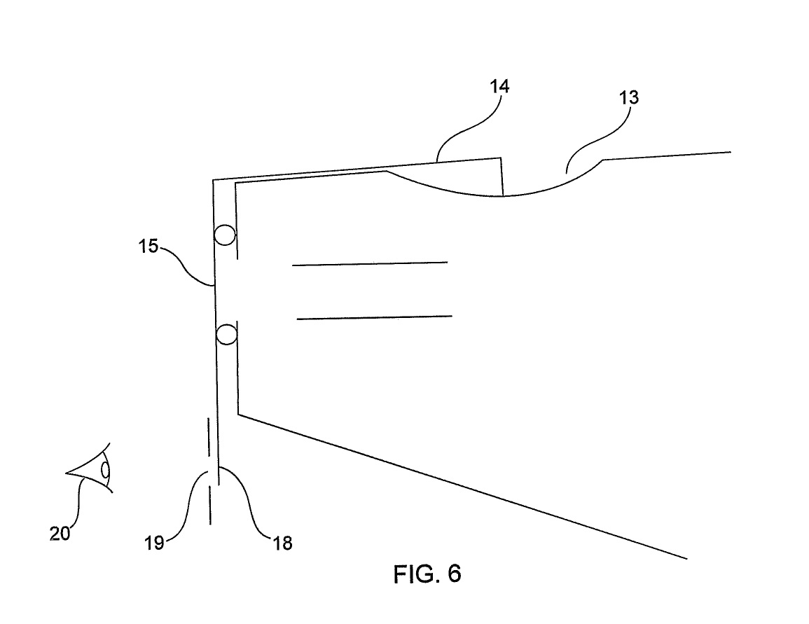

FIG. 6 shows an embodiment of the invention wherein the users is notified that the sterility may have been compromised and he/she should not use the device. When the pressure drops below a pre-determined third pressure which is less than the second pressure, diaphragm 13 moves cover 15 to a location such that a target, flag or marking 18 is visible through window 19. The flag could be any color, although the colors red, orange, or yellow are preferred. Many other ways of alerting the user could be used, including a pressure transducer and electronics that activate a signal such as a light or sound.

### Example 1

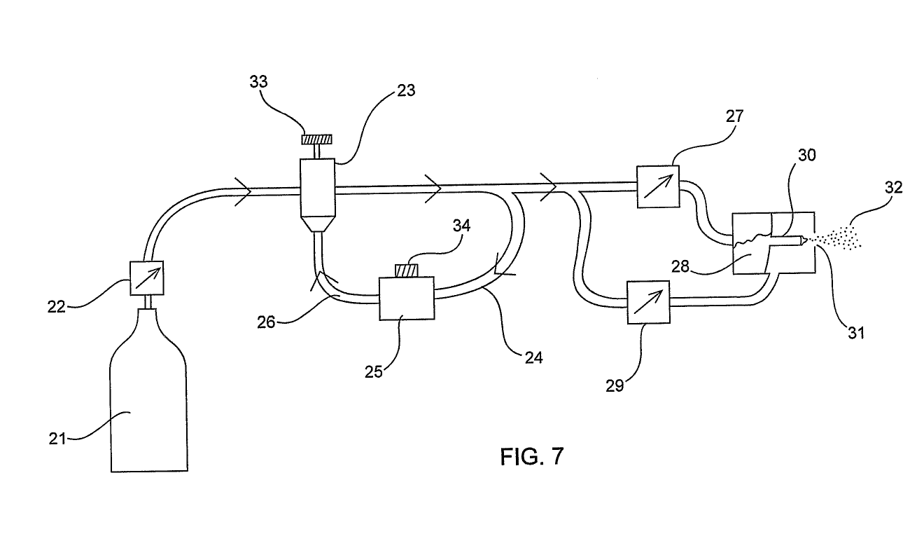

A system was developed to use gas to meter out a formulation, and then used the same gas to generate an aerosol (FIG. 7). In this case, the gas was air, contained within an external tank (21). The gas delivered to the system was regulated by a pressure regulator (22) to 60 PSI. The gas is then delivered to a pneumatic switch (Kuhnke part number 75.022.27.22) (23). When the button (33) on the switch (23) was depressed, gas flowed to the pneumatic timer (Kuhnke part #51.006.00) (25) via a tube (24). The timer (25) was set using a knob (34) to 22 seconds. After 22 seconds, the timer (25) allowed the gas to flow though a tube (26) to the switch (23) turning off the flow of gas thereby venting the system for rapid turn-off. During the 22 seconds the gas was on, the formulation (28) was pressurized to 35 PSI, said 35 PSI being controlled by a regulator (27). Also, the aerosolization gas flow pressure was controlled at 30 PSI by a regulator (29). The pressurized formulation (28) was forced though the capillary (30) and the gas and liquid flowed out of the orifice (31) to form the aerosol (32). Not shown are pressure transducers to measure the aerosolization gas pressure and formulation pressure, and a differential pressure transducer across the capillary (30) to measure the liquid flow.

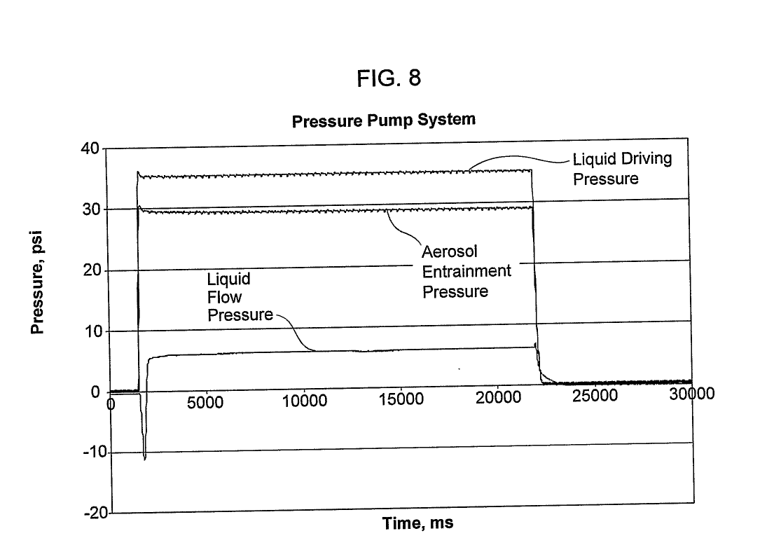

The results are shown in FIG. 8. The gas pressure, liquid pressure, and gas flow rate (arbitrary units) are all controlled to give a duration of aerosol generation of ˜22 seconds.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A drug delivery device, comprising:
a container of pressurized gas;
a component which releases a metered amount of gas from the container on activation;
a reservoir which holds a formulation of pharmaceutically active drug;
a channel in fluid connection with the reservoir; and
a chamber in physical contact with the reservoir and in gas flow connection with the container of pressurized gas such that when pressurized gas is released from the container to the chamber the reservoir is compressed and formulation expelled from the channel at a rate of delivery.

2. The drug delivery device of claim 1 wherein the component is a metering valve and the rate of delivery of the formulation is in the range of 0.1 to 500 μL/s.

3. The drug delivery device of claim 2 wherein the rate of delivery of the formulation is in the range of 1 to 250 μL/s.

4. The drug delivery device of claim 3 wherein the rate of delivery of the formulation is in the range of 3 to 100 μL/s.

5. The drug delivery device of claim 1, further comprising:
a mechanical linkage in physical contact with a diaphragm component of the chamber so that when the chamber is pressurized the mechanical linkage opens a sealed area surrounding the reservoir and channel.

6. The drug delivery device of claim 1, where in the pressurized gas of the container is pressurized to a liquid state and the liquid is chosen from CO₂, N₂O and a hydro-fluoro-alkane.

7. The drug delivery device of claim 6, wherein the pressurized gas in a liquid state is present in an amount of from about 2 grams to about 50 grams.

8. The drug delivery device of claim 1 wherein said reservoir is comprised of a flexible material chosen from, polyethelene, Cyclo Olefin Copolymers (COCs), Polychlorotrifluoroethylene, Chlorotrifluoroethene (PCTFE), Aluminum, Nylon, and Polyester.

9. (canceled)

10. The drug delivery device of claim 2, further comprising:
a mouthpiece positioned in a direction of outward flow relative to the channel.

11. The drug delivery device of claim 1, wherein the channel is a right circular cylinder.

12. The drug delivery device of claim 11, wherein the cylinder has a cross sectional area of from about 0.01 to 0.05 mm² and a length of about 1 mm to about 12 mm.

13. A drug delivery device, comprising:
a docking unit for attachment of a pressurized gas container comprising a metering valve;
a reservoir which holds a formulation of pharmaceutically active drug;
a channel in fluid connection with the reservoir; and
a chamber in physical contact with the reservoir and in gas flow connection with the docking unit.

14. The device delivery device of claim 13, further comprising:
a pressurized gas container connected to the docking unit.

15. The drug delivery device of claim 13, further comprising:
a one way valve in the channel allowing flow out of but not into the reservoir.

16. The drug delivery device of claim 13, further comprising:
a mechanical linkage in connection with a moveable component of the chamber so that when the chamber is pressurized the movable component moves the mechanical linkage so as to open a sealed area surrounding the reservoir and channel.

17. The drug delivery device of claim 13, further comprising:
a lock-out linkage in connection with the chamber positioned and structured so that when the chamber pressure drops below a predetermined level the lock-out linkage seals an area in a manner so as to prevent delivery of drug from the channel.

18. The drug delivery device of claim 13, further comprising:
a sterility breach warning linkage in connection with the chamber positioned and structured so that when the chamber pressure drops below a predetermined level the warning linkage moves to show a sterility breach warning signal.

19. A method of maintaining drug sterility, comprising:
releasing pressurized gas from a canister and into a chamber;
changing pressure in the chamber from a first pressure to a second pressure amount so as to displace a movable component connected to the chamber;
forcing a sealing component in a direction relative to an exit orifice so as to control discharge of a drug formulation from a reservoir of drug formulation.

20. The method of claim 19, wherein the change in pressure is a decrease and the sealing component controls discharge by preventing discharge.

21. A method of maintaining sterility in a drug reservoir, comprising:
delivering a liquid formulation at a first pressure;
storing the liquid formulation at a second pressure;
wherein said second pressure is greater than the surrounding, ambient pressure.

22. The method of claim 21, wherein said second pressure is less than 50 bar.

23. The method of claim 22 wherein said second pressure is less than 10 bar.

24. The sterile reservoir of claim 23 wherein said second pressure is less than 5 bar.

25. The drug delivery device, comprising:
a pneumatic timer;
a mechanism for delivering formulation from a multi dose reservoir to an atomizer;
a capillary for delivering the formulation to the atomizer; and
a one way valve configured such that the formulation can flow to the atomizer when the formulation is pressurized to a first pressure;
wherein the one way valve closes at a second pressure below said first pressure.

26. The mechanism of claim 25, wherein the formulation is a liquid formulation.
